(19) United States
(12) Patent Application Publication
(10) Pub. No.: US 2021/0180107 A1
(43) Pub. Date: Jun. 17, 2021

(54) OLIGONUCLEOTIDES AND METHOD FOR THE DETECTION AND TYPING OF HUMAN PAPILLOMAVIRUS

(71) Applicants: Universidade Federal de Uberlandia, Uberlândia - MG (BR); Fundação de Amparo a Pesquisa do Estado de Minas Gerais - Fapemig, Belo Horizonte - MG (BR)

(72) Inventor: Luiz Goulart, Uberlândia - MG (BR)

(21) Appl. No.: 16/492,032

(22) PCT Filed: Mar. 8, 2018
(86) PCT No.: PCT/BR2018/050062

§ 371 Date: Dec. 1, 2020

(30) Foreign Application Priority Data
Mar. 8, 2017 (BR) 10 2017 004615 0

(51) Int. Cl. C12Q 1/686; C12Q 1/6806; C12Q 1/6876

(52) U.S. Cl. CPC C12Q 1/686; C12Q 1/6806; C12Q 2537/143; C12Q 2563/173; C12Q 2565/125; C12Q 1/6876

(57) ABSTRACT

The present invention relates to the use of nucleic acid amplification technology through two-step (duplicated) polymerase chain reactions with multiple markers, also known as multiplex nested PCR, using general consensus oligonucleotides followed by multiple specific oligonucleotides and the components thereof (reagents and reaction conditions) to simultaneously detect 40 types of human papillomavirus present in fluids and samples of human tissue, and also carry-out a semi-quantitative analysis between viral types, determining the viral dominance relationship (viral load) in multiple infections. The sequences of the general consensus-degenerate primers (specific initiators or oligonucleotides, SEQ ID NOS: 1 to 93) (1^{st} PCR reaction) and combined primers are disclosed, as well as specific combined primers (2^{nd} nested PCR reaction) for 40 viral types, which include non-oncogenic, indeterminate and oncogenic types.

### FIELD OF THE INVENTION

The present invention relates to the identification of oligonucleotides and to a method for detecting and typing human papillomavirus. This method relates to the use of nucleic acid amplification technology by two-step (duplicated) polymerase chain reaction with multiple markers, also called multiplex nested PCR, using general consensus oligonucleotides followed by multiple specific oligonucleotides and their components (reagents and reaction conditions) to simultaneously detect 40 human papillomavirus types present in fluids and human tissue samples, as well as to perform a semiquantitative analysis among the viral types, determining the viral dominance relationship (viral load) in multiple infections. The sequences of the general consensus-degenerate primers (specific initiators or oligonucleotides) (1^{st} PCR reaction) and combined primers, as well as the specific combined primers (2^{nd} nested PCR reaction) for the viral types, which encompass non-oncogenic, indeterminate and oncogenic types (6, 11, 16, 18, 26, 30, 31, 33, 34, 35, 39, 42, 43, 44, 45, 51, 52, 53, 54, 55, 56, 57, 58, 59, 61, 62, 64, 66, 67, 68, 69, 70, 71, 72, 73, 74, 81, 82, MM7, and MM8), are described and are part of the invention that utilizes the multiplex nested PCR for the simultaneous typing of 40 viral types in a single tube. The detection process is performed by fluorescence in sequencers by capillary electrophoresis, which also allows the semiquantitative determination of the viral load of viral types in multiple infections. Additionally, the invention can be used to monitor the treatment of HPV infections in both men and women.

### STATE OF THE ART

HPV has been implicated in the genesis of several pre-cancerous and neoplasic lesions, particularly of uterine cervix carcinomas, anogenital region, skin, respiratory and digestive tract. HPV research is recommended in the following cases: women with atypical or borderline colpocytology, high-risk patients (early sexual initiation with several partners), immunocompromised individuals and partners of HPV-infected patients.

HPV can be classified in approximately 100 different epitheliotropic types, and about 40 types are exclusively mucosotrophic. Approximately one third of these mucosotrophic HPV types were isolated from cervical carcinomas (HO GYF, BIRMAN R, BEARDSLAY L et al. 1998. New England J. Med. 338, pages 423-428).

The PCR method (U.S. Pat. Nos. 4,683,195 and 5,639,871) was introduced as the most sensitive method for detecting HPV DNA (deoxyribonucleic acid) in clinical specimens. The first molecular diagnoses, using the polymerase chain reaction (PCR), were developed at the end of the 80's (MANOS M M, TING Y, WRIGHT D K et al. 1989. Cancer Cell 7, pages 209-214). Few HPVs had their genome sequenced and, despite this, a wide genetic variability was observed, which implies that, even using regions conserved among viruses, their detection is difficult. This viral variability has stimulated the development of simple and universal tests using PCR (polymerase chain reaction) allowing the detection of a broad spectrum of the main HPV genotypes (MANOS M M, TING Y, WRIGHT D K et al. 1989. Cancer Cell 7, pages 209-214; SNIJDERS P J F., VAN DE BRULE A J F, SCHRIJNEMAKERS H J F et al. 1990. J. Gen. Virol. 71, pages 173-181).

Many patents have been described searching for HPV typing, using distinct or similar genomic regions, or using a mixture of techniques such as PCR and hybridization, and also using differentiated targets, which can be either DNA or RNA. Among the various publications and relevant patents some are cited below, making a comparison with the present invention:

JP2012075437A refers to the description of 13 pairs of primers (probes) for multiplex detection of 13 high-risk viral types only. Detection occurs by conventional electrophoresis and does not use nested (duplicated) PCR. The difference with the present invention lies in detecting 40 viral types with 40 pairs of different primers in nested PCR, which includes a first general amplification and a second specific duplicate multiplex amplification. Furthermore, the detection proposed in this invention is much superior for being simultaneous in a single reaction with capillary electrophoresis by laser fluorescence.

Patent DE19903056A1 refers to the detection of viral types by nested PCR. It uses 4 primers in the first reaction and 8 probes in the second, and claim that they detect approximately 30 viral types, specifying only 6 viral types (6, 11, 16, 18, 31, 33), with the last 4 classified as being of high risk. Detection is carried out by hybridization with two specific probes in membrane blots (strips). It is different from the present invention, that uses a primary amplification with several degenerated general primers (10), plus three primers as internal control (beta globin). The second reaction occurs simultaneously with more 40 pairs of specific primers with laser fluorescence detection in capillary electrophoresis, and not by hybridization, which makes the present technology more competitive and of high performance, with less manipulation.

Patent KR2004047238A seeks protection only to sequences of three sets of primer pairs, 6 in all, for the general detection of various types of HPV unspecifically by conventional PCR, and there are common amplifications among them, probably due to the great viral variability. The detection method is not elaborated. There is no similarity to the present invention.

Patent CN102229930A uses a multiplex real-time PCR technology to detect 18 high-risk viral types. The patent describes 18 pairs of primers and their associated probes, being a different technology of nested PCR with simultaneous detection of 40 viral types by fluorescence claimed in the present invention, with comparable sensitivity. However, the referred patent requires several multiplex, as real-time PCR has a maximum of 4 possible fluorophores, allowing simultaneous detection of only 4 types at a time, therefore not presenting the high performance claimed by the present invention that carries out the test in a single tube for typing 40 viral types.

Patent U.S. Pat. No. 6,482,588 B1 describes two amplified regions with 3 primers (SGP1, SGP2 and SGP3), the latter being common to the end of the region amplified by the primers MY11/MY09, that is, outside the region of the present invention, and uses a different platform known as LiPA, or also reverse slot-blot. The probes used in this system are smaller and very different from those used in the present invention.

Patent WO 2008/089519 A1 seeks protection to the detection of 17 viral types by real-time PCR with multiplex of 4 sets of primers. The patent also seeks protection to two primers for general detection (screening), demonstrating to be superior to the “hybrid Capture 2” diagnostic system. No sequence of the claimed primers coincides with the primers of the present invention; nevertheless, the technology claimed in that patent requires multiple primer panels, while the present invention proposes amplification of 40 virus types in a single tube.

Another system similar to the previous patent was developed for quantifying seven high-risk viral types by multiplex real-time PCR (SCHMITZ M, SCHEUNGRABER C, HERRMANN J et al. 2009. J. Clin. Virol, 44, p. 302-307). This technique uses 7 pairs of primers and seven probes, which have very different sequences from the present invention, that detects 40 viral types by another technology.

Patent WO 2011/088573 A1 was filed recently, nine years later than the patent that originated the present invention, which detected 32 viral types with 32 pairs of primers (PI0302987-5, of Jul. 16, 2003). WO 2011/088573 A1 is based on amplification with the pair of primers GP5+/GP6+(SNIJDERS P J F, VAN DE BRULE A J F, SCHRIJNEMAKERS H J F et al. 1990. J. Gen. Virol. 71, pages 173-181; Patent WO 91/10675), but with detection by hybridization with 46 probes in the Luminex system, which is based on flow cytometry with fluorescent microspheres. The present invention is based on amplification by nested PCR with fluorescent detection in a capillary electrophoresis system. It is important to emphasize that these sequences function as hybridization probes in the Luminex equipment, while the present invention refers to one of the primers of the pair that recognizes the viral type by nested PCR and fluorescence detection in capillary electrophoresis.

However, the frequency of lesions with false-negative results for the presence of HPV has increased, suggesting that a substantial part of viral types is not being detected and, therefore, that epidemiological studies as well as actions for prevention and treatment have been impaired.

In order to seek a methodology capable of performing fast, efficient and more sensitive typing of human papillomaviruses that encompass at least the 40 most frequent viral types associated with cervical and penile lesions, among the more than 100 types already described, we developed a PCR variant technique (patent U.S. Pat. Nos. 4,683,195 and 5,639,871), nested PCR, with multiple simultaneous primers (also called multiplex PCR), described for the first time in 1994 (Patent U.S. Pat. No. 5,364,759) for simultaneous genotyping of STRs (consecutive short DNA repeats). The present invention presents this technique associated with a capillary electrophoresis equipment with fluorescence detection applied for simultaneously detecting 40 HPV virus types in a single tube, encompassing the most frequent types in fresh or paraffin preserved samples of the cervix, anogenital region, skin, respiratory and digestive tract.

It is important to note that the present invention also provided the determination of the relative viral load among the infective viruses in a sample, after being submitted to a series of dilutions, thus allowing the verification of the viral dominance in the lesion and enabling the adoption of specific criteria for treatment and clinical observations according to the dominant virus type. In case of a high risk dominant type, such as HPV16, the risk of cancer in situ is often increased, subsidizing the therapeutic and prophylactic measures to be adopted.

### LIST OF FIGURES

The invention can be better understood through the detailed description, in accordance with the attached figures, where:

FIG. 1 schematically represents the large-scale commercial platform of multiplex nested PCR used to simultaneously detect 40 HPV types in a single sample, in a two-step assay with an average duration of 6 hours.

FIG. 2 demonstrates the data used to construct a new PeakFilter in the Fragment Profiler software of the capillary automatic sequencer (MegaBace 1000) specific for each HPV type.

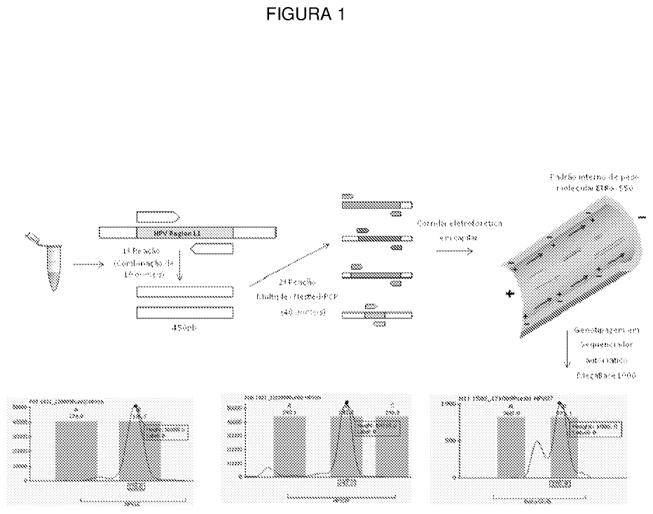

Referring to FIG. 1, the commercial platform of multiplex nested PCR is extremely specific, with the use of specific tagged primers, where the 40 types of HPV separately amplified are subjected to electrophoretic analysis in an automatic sequencer. The internal molecular weight standard ETRox-550 (GE Healthcare) is used in the sample analysis. The beta globin gene present in all assays, is used as an amplification control.

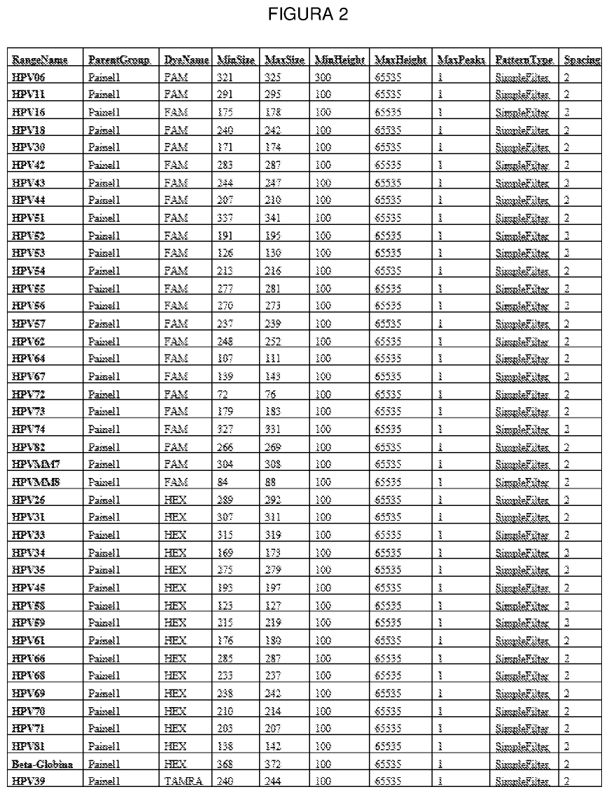

Referring to FIG. 2, a new PeakFilter was built in the Fragment Profiler software to allow electrophoretic reading and data analysis, contemplating the three fluorescent markings (FAM, HEX and TAMRA).

### DESCRIPTION OF THE INVENTION

Below is a detailed description of the invention for better understanding the proposed method.

Viral DNA is obtained from cervical cells, penile scratch, anus, mouth, skin and paraffin blocks, among other human samples (GRIFFITH et al. 2001. Molecular Cloning: A Laboratory Manual, vols. 1, 2 and 3). The fragment of the 1st PCR reaction that amplifies a specific gene region of HPV (L1) is used as the basis for the degeneration and the design and synthesis of new markers, this sequence amplified by the degenerate primers MY09 and MY11 that constitute part of the U.S. Pat. No. 5,364,758. In this regard, 10 primers were developed, 5 of DNA sense and 5 of DNA antisense, forming 25 combinations of primer pairs. These primer combinations can detect the presence of viruses in unlayered cells of any organic sample to be tested, and can detect all HPV types described in the literature, based on the sequences deposited in GenBank (http://www.ncbi.nlm.nih.gov/GenBank). Virus typing is obtained from a second nested (or duplicated) reaction, which is performed from 40 specific primer pairs. The primers were combined in a multiplex reaction in a single tube allowing the specific analysis of 40 HPV virus types.

The HPV amplification reactions were performed in two stages, the first with the use of ten degenerate and modified primers (SEQ ID NOS: 01, 02, 03, 04, 05, 06, 07, 08, 09 and 10) generating a fragment with 450 base pairs (PCR-OUT). This reaction is performed to detect the virus in patients, amplifying a region common to the 40 types of papillomavirus. It consists of 1× Taq Platinum buffer, 25-30 pmol of each degenerate and modified consensus primer, 1.5 U Taq DNA polymerase, 200 μM dNTPs, 2 pmol of each primer of the internal control gene of beta globin (SEQ ID NOS: 11 and 12) and 7 μL of DNA to a final volume of 30 μL. The reaction conditions were: 40 cycles of 94° C. for 1 min., 50° C. for 1 min., 72° C. for 2 min. and final extension at 72° C. for 5 min. and 4° C. for 5 min.

In the second stage, 2 μL of the amplified product in the first reaction were used as template in each mix, according to the previously standard Multiplex-Nested reaction. This reaction consists of 1× taqPhoneutria buffer, 0.5 pmol of specific primers (SEQ ID NOS: 14-93) for the 40 types of papillomavirus, 0.2 pmol of each primer of the constitutive beta-globin gene (SEQ ID Nos: 12 and 13), 1.0 U Taq DNA polymerase, 200 μM of dNTPs, 1× enhancer to a final volume of 15 μL. The reaction conditions were: 95° C. for 1 min., 36 cycles of 95° C. for 1 min., 56° C. for 1 min., 72° C. for 40 s., and final extension at 72° C. for 5 min. and 4° C. for 5 min. Two microliters of the amplified product are subjected to capillar electrophoretic analysis in the MegaBace 1000® sequencer (GE Healthcare), using the molecular weight internal standard ET550-R (GE Healthcare) for the sample analysis. As amplification control, the beta globin gene, present in all assays, is used.

The oligonucleotide identity and sequences (5′-3′ sense) used for amplifying the two PCR sequence reactions (Multiplex Nested) are found below. The sequences are divided into general (1st reaction) and specific (2^{nd} reaction) oligonucleotides, totaling SEQ ID NO: 01 to SEQ ID NO: 93.

1. An oligonucleotide comprising one of the sequences of SEQ ID NO: 01 to SEQ ID NO: 93.

2. The oligonucleotide, according to claim 1, wherein in SEQ ID NO: 01 to SEQ ID NO: 13 participate in the first phase of the PCR reaction, generating a fragment with 450 base pairs.

3. The oligonucleotide, according to claim 1, wherein in that SEQ ID NO: 14 to SEQ ID NO: 93 participate in the second stage of the Multiplex nested PCR reaction in a single reaction tube for capillary electrophoresis detection.

4. A method for detecting and typing human papillomavirus, comprising the combined use of the two-step (duplicate) multiplex nested PCR technique with multiple markers using general consensus oligonucleotides (first stage) followed by multiple specific oligonucleotides (second stage) and their components (reagents and reaction conditions) for simultaneously detecting 40 human papillomavirus types present in fluids and human tissue samples, in a single reaction tube for capillary electrophoresis detection.

5. The method, according to claim 4, comprising detecting the viral type(s) in a simple agarose system stained with ethidium bromide, in an acrylamide system stained with silver, or even in a multiple or simple fluorescence system in a capillary electrophoresis system for simultaneously detecting amplicons obtained with primers tagged with fluorophores.

6. The method, according to claim 4, wherein the detection of at least 40 more frequent viral types with initial PCR amplification of a sequence from the L1 genomic region of HPV containing 10 (ten) general primers with consensus sequences representative of HPV described by SEQ ID NO: 01 to SEQ ID NO: 10, which will also be a mold for the second stage of the PCR (Nested PCR) reaction.

7. The method, according to claim 4, wherein the specific simultaneous reamplification of 40 viral types with internal oligonucleotides (multiplex nested PCR) homologous to the sequences of specific amplicons generated in the 1^{st} PCR reaction.

8. The method, according to claim 4, wherein the probes or primers described in SEQ ID NO: 01 to SEQ ID NO: 93 can detect oncogenic, non-oncogenic and indeterminate types as to the risk of developing neoplasic lesion.

9. The method, according to claim 4, wherein the general and specific oligonucleotides, described in claims 1, 2, 3, and by the detection methods described in claim 5, to be used in the isolated or multiple detection of the HPV types, in any combination of primers.


SEQUENCE LISTING (93 sequences)

SEQ ID NO: 1 - DNA, 20 bp, Artificial Sequence
  Synthesized oligonucleotide - primer for HPV
  misc_feature (3)..(3): n is a, c, g, or t
ckncchardggaaactgatc

SEQ ID NO: 2 - DNA, 20 bp, Artificial Sequence
  Synthesized oligonucleotide - primer for HPV
cgtccmaarggahactgatc

SEQ ID NO: 3 - DNA, 20 bp, Artificial Sequence
  Synthesized oligonucleotide - primer for HPV
cgtccmarrggatactgatc

SEQ ID NO: 4 - DNA, 20 bp, Artificial Sequence
  Synthesized oligonucleotide - primer for HPV
  misc_feature (3)..(3): n is a, c, g, or t
gcncaggghcataacaatgg

SEQ ID NO: 5 - DNA, 20 bp, Artificial Sequence
  Synthesized oligonucleotide - primer for HPV
  misc_feature (3)..(3): n is a, c, g, or t
gcncaggghcacaataatgg

SEQ ID NO: 6 - DNA, 20 bp, Artificial Sequence
  Synthesized oligonucleotide - primer for HPV
cchcargghcataataatgg

SEQ ID NO: 7 - DNA, 20 bp, Artificial Sequence
  Synthesized oligonucleotide - primer for HPV
ckmccharkggawtatgatc

SEQ ID NO: 8 - DNA, 20 bp, Artificial Sequence
  Synthesized oligonucleotide - primer for HPV
ckdccyardggraattggtc

SEQ ID NO: 9 - DNA, 20 bp, Artificial Sequence
  Synthesized oligonucleotide - primer for HPV
  misc_feature (3)..(3): n is a, c, g, or t
gcncaggghcabaacaatgg

SEQ ID NO: 10 - DNA, 20 bp, Artificial Sequence
  Synthesized oligonucleotide - primer for HPV
gcycaggghywmaacaatgg

SEQ ID NO: 11 - DNA, 22 bp, Artificial Sequence
  Synthesized oligonucleotide - primer
cacctttgccacactgagtgag

SEQ ID NO: 12 - DNA, 23 bp, Artificial Sequence
  Synthesized oligonucleotide - primer
agtaatgtactaggcagactgtg

SEQ ID NO: 13 - DNA, 21 bp, Artificial Sequence
  Synthesized oligonucleotide - primer
aagctgcacgtggatcctgag

SEQ ID NO: 14 - DNA, 20 bp, Artificial Sequence
  Synthesized oligonucleotide - primer for MM7
ggatcctttttaggggcagg

SEQ ID NO: 15 - DNA, 23 bp, Artificial Sequence
  Synthesized oligonucleotide - primer for MM7
agtaccaatattactatttcagc

SEQ ID NO: 16 - DNA, 20 bp, Artificial Sequence
  Synthesized oligonucleotide - primer for HPV type 59
aagttacagcagcagattga

SEQ ID NO: 17 - DNA, 23 bp, Artificial Sequence
  Synthesized oligonucleotide - primer for HPV type 59
cctaccagttttaaagaatatgc

SEQ ID NO: 18 - DNA, 20 bp, Artificial Sequence
  Synthesized oligonucleotide - primer for HPV type 44
taaggtaccatttgggggcg

SEQ ID NO: 19 - DNA, 20 bp, Artificial Sequence
  Synthesized oligonucleotide - primer for HPV type 44
taaggtaccatttgggggcg

SEQ ID NO: 20 - DNA, 21 bp, Artificial Sequence
  Synthesized oligonucleotide - primer for HPV type 43
catgcaatggccttgttagac

SEQ ID NO: 21 - DNA, 19 bp, Artificial Sequence
  Synthesized oligonucleotide - primer for HPV type 43
ctgaccctactgtgcccag

SEQ ID NO: 22 - DNA, 22 bp, Artificial Sequence
  Synthesized oligonucleotide - primer for HPV type 30
ggtgacaatccaatattccagc

SEQ ID NO: 23 - DNA, 24 bp, Artificial Sequence
  Synthesized oligonucleotide - primer for HPV type 30
cgttatccacatataattcaagcc

SEQ ID NO: 24 - DNA, 21 bp, Artificial Sequence
  Synthesized oligonucleotide - primer for HPV type 67
cataacatttgcagtaaggga

SEQ ID NO: 25 - DNA, 22 bp, Artificial Sequence
  Synthesized oligonucleotide - primer for HPV type 67
aacatgactttatattctgagg

SEQ ID NO: 26 - DNA, 22 bp, Artificial Sequence
  Synthesized oligonucleotide - primer for HPV type 64
tccactgttctaatatactaga

SEQ ID NO: 27 - DNA, 20 bp, Artificial Sequence
  Synthesized oligonucleotide - primer for HPV type 64
tgcagaagagtatgacctcg

SEQ ID NO: 28 - DNA, 20 bp, Artificial Sequence
  Synthesized oligonucleotide - primer for HPV type 26
ctgacaggtagtagcagagt

SEQ ID NO: 29 - DNA, 21 bp, Artificial Sequence
  Synthesized oligonucleotide - primer for HPV type 26
accacccgcagtactaacctt

SEQ ID NO: 30 - DNA, 23 bp, Artificial Sequence
  Synthesized oligonucleotide - primer for HPV type 34
tccactgttccaatatactagaa

SEQ ID NO: 31 - DNA, 21 bp, Artificial Sequence
  Synthesized oligonucleotide - primer for HPV type 34
acaatccacaagtacaactgc

SEQ ID NO: 32 - DNA, 23 bp, Artificial Sequence
  Synthesized oligonucleotide - primer for HPV type 66
cccaaaacttatatttagccagg

SEQ ID NO: 33 - DNA, 23 bp, Artificial Sequence
  Synthesized oligonucleotide - primer for HPV type 66
ctaaatatgatgcccgtgaaatc

SEQ ID NO: 34 - DNA, 23 bp, Artificial Sequence
  Synthesized oligonucleotide - primer for HPV type 62
gagactcgaaatagtgatatgtc

SEQ ID NO: 35 - DNA, 23 bp, Artificial Sequence
  Synthesized oligonucleotide - primer for HPV type 62
ctaattttactatttgtaccgcc

SEQ ID NO: 36 - DNA, 20 bp, Artificial Sequence
  Synthesized oligonucleotide - primer for HPV type 57
aatacctgtaggtgtcctgc

SEQ ID NO: 37 - DNA, 23 bp, Artificial Sequence
  Synthesized oligonucleotide - primer for HPV type 57
gtctctttgtgtgccactgtaac

SEQ ID NO: 38 - DNA, 19 bp, Artificial Sequence
  Synthesized oligonucleotide - primer for HPV type 55
ctttgccttttcaggggga

SEQ ID NO: 39 - DNA, 20 bp, Artificial Sequence
  Synthesized oligonucleotide - primer for HPV type 55
gctgctacaactcagtctcc

SEQ ID NO: 40 - DNA, 20 bp, Artificial Sequence
  Synthesized oligonucleotide - primer for HPV type 6
cctcccaaaaactaaggttc

SEQ ID NO: 41 - DNA, 21 bp, Artificial Sequence
  Synthesized oligonucleotide - primer for HPV type 6
acatgacattatgtgcatccg

SEQ ID NO: 42 - DNA, 18 bp, Artificial Sequence
  Synthesized oligonucleotide - primer for HPV type 16
tcttctagtgtgcctcct

SEQ ID NO: 43 - DNA, 21 bp, Artificial Sequence
  Synthesized oligonucleotide - primer for HPV type 16
ctaactttaaggagtacctac

SEQ ID NO: 44 - DNA, 20 bp, Artificial Sequence
  Synthesized oligonucleotide - primer for HPV type 31
caattgcaaacagtgatact

SEQ ID NO: 45 - DNA, 21 bp, Artificial Sequence
  Synthesized oligonucleotide - primer for HPV type 31
acctcccaaaatacataatct

SEQ ID NO: 46 - DNA, 21 bp, Artificial Sequence
  Synthesized oligonucleotide - primer for HPV type 33
tttatgcacacaagtcactag

SEQ ID NO: 47 - DNA, 24 bp, Artificial Sequence
  Synthesized oligonucleotide - primer for HPV type 33
cacttcccaaaatgtatatttacc

SEQ ID NO: 48 - DNA, 20 bp, Artificial Sequence
  Synthesized oligonucleotide - primer for HPV type 35
tgttctgctgtgtctwctag

SEQ ID NO: 49 - DNA, 18 bp, Artificial Sequence
  Synthesized oligonucleotide - primer for HPV type 35
ggttttggtgcactgggt

SEQ ID NO: 50 - DNA, 20 bp, Artificial Sequence
  Synthesized oligonucleotide - primer for HPV type 39
tacctctatagagtcttcca

SEQ ID NO: 51 - DNA, 22 bp, Artificial Sequence
  Synthesized oligonucleotide - primer for HPV type 39
agactgtaagtatctgtaagtg

SEQ ID NO: 52 - DNA, 19 bp, Artificial Sequence
  Synthesized oligonucleotide - primer for HPV type 45
gaaatcctgtgccargtac

SEQ ID NO: 53 - DNA, 19 bp, Artificial Sequence
  Synthesized oligonucleotide - primer for HPV type 45
tgtagtaggtggtggaggg

SEQ ID NO: 54 - DNA, 19 bp, Artificial Sequence
  Synthesized oligonucleotide - primer for HPV type 51
actattagcactgccactg

SEQ ID NO: 55 - DNA, 23 bp, Artificial Sequence
  Synthesized oligonucleotide - primer for HPV type 51
aatcgttcctttaaatcaacatc

SEQ ID NO: 56 - DNA, 20 bp, Artificial Sequence
  Synthesized oligonucleotide - primer for HPV type 52
ctgaggtkaaaaaggaaagc

SEQ ID NO: 57 - DNA, 17 bp, Artificial Sequence
  Synthesized oligonucleotide - primer for HPV type 52
acggtggtggggtaagg

SEQ ID NO: 58 - DNA, 18 bp, Artificial Sequence
  Synthesized oligonucleotide - primer for HPV type 53
ttccgcaaccacacagtc

SEQ ID NO: 59 - DNA, 18 bp, Artificial Sequence
  Synthesized oligonucleotide - primer for HPV type 53
taacctcagcagacaggg

SEQ ID NO: 60 - DNA, 20 bp, Artificial Sequence
  Synthesized oligonucleotide - primer for HPV type 54
gtgtgctacagcatccacgc

SEQ ID NO: 61 - DNA, 20 bp, Artificial Sequence
  Synthesized oligonucleotide - primer for HPV type 54
tcctccaaactacttgtagc

SEQ ID NO: 62 - DNA, 19 bp, Artificial Sequence
  Synthesized oligonucleotide - primer for HPV type 62
tagtactgctacagaacag

SEQ ID NO: 63 - DNA, 17 bp, Artificial Sequence
  Synthesized oligonucleotide - primer for HPV type 56
tttggtggctgttcccg

SEQ ID NO: 64 - DNA, 20 bp, Artificial Sequence
  Synthesized oligonucleotide - primer for HPV type 61
atttgtactgctacatcccc

SEQ ID NO: 65 - DNA, 18 bp, Artificial Sequence
  Synthesized oligonucleotide - primer for HPV type 61
gagtcatccaacaaggcc

SEQ ID NO: 66 - DNA, 20 bp, Artificial Sequence
  Synthesized oligonucleotide - primer for HPV type 68
tactactactgaatcagctg

SEQ ID NO: 67 - DNA, 18 bp, Artificial Sequence
  Synthesized oligonucleotide - primer for HPV type 68
actgattgcagatagcgg

SEQ ID NO: 68 - DNA, 20 bp, Artificial Sequence
  Synthesized oligonucleotide - primer for HPV type 70
gccatacctgctgtatatag

SEQ ID NO: 69 - DNA, 19 bp, Artificial Sequence
  Synthesized oligonucleotide - primer for HPV type 70
cctatacgtgtccactaag

SEQ ID NO: 70 - DNA, 20 bp, Artificial Sequence
  Synthesized oligonucleotide - primer for MM8
aggaatacctaagacatgtg

SEQ ID NO: 71 - DNA, 18 bp, Artificial Sequence
  Synthesized oligonucleotide - primer for MM8
catgacctctggagtcag

SEQ ID NO: 72 - DNA, 17 bp, Artificial Sequence
  Synthesized oligonucleotide - primer for HPV type 72
tctcctgtacctgggca

SEQ ID NO: 73 - DNA, 20 bp, Artificial Sequence
  Synthesized oligonucleotide - primer for HPV type 18
ggtaatagcaacagattgtg

SEQ ID NO: 74 - DNA, 20 bp, Artificial Sequence
  Synthesized oligonucleotide - primer for HPV type 11
catctgtgtctaaatctgct

SEQ ID NO: 75 - DNA, 20 bp, Artificial Sequence
  Synthesized oligonucleotide - primer for HPV type 11
tatccttatagggatcctgt

SEQ ID NO: 76 - DNA, 21 bp, Artificial Sequence
  Synthesized oligonucleotide - primer for HPV type 58
cactgaagtaactaaagaaga

SEQ ID NO: 77 - DNA, 21 bp, Artificial Sequence
  Synthesized oligonucleotide - primer for HPV type 58
cattacctctgcagttagtgt

SEQ ID NO: 78 - DNA, 21 bp, Artificial Sequence
  Synthesized oligonucleotide - primer for HPV type 72
actatttgtactgccacagcg

SEQ ID NO: 79 - DNA, 20 bp, Artificial Sequence
  Synthesized oligonucleotide - primer for HPV type 72
gtgtggcgaagatactcacg

SEQ ID NO: 80 - DNA, 21 bp, Artificial Sequence
  Synthesized oligonucleotide - primer for HPV type 81
ctatttgcacagctacatctg

SEQ ID NO: 81 - DNA, 21 bp, Artificial Sequence
  Synthesized oligonucleotide - primer for HPV type 81
gtaggccataatttctggtgt

SEQ ID NO: 82 - DNA, 23 bp, Artificial Sequence
  Synthesized oligonucleotide - primer for HPV type 73
attccactcttccaatatagtag

SEQ ID NO: 83 - DNA, 20 bp, Artificial Sequence
  Synthesized oligonucleotide - primer for HPV type 73
gtaggtacacaggctagtag

SEQ ID NO: 84 - DNA, 22 bp, Artificial Sequence
  Synthesized oligonucleotide - primer for HPV type 71
ctgtgctaccaaaactgttgag

SEQ ID NO: 85 - DNA, 20 bp, Artificial Sequence
  Synthesized oligonucleotide - primer for HPV type 71
agcagtaggaggtggtaagg

SEQ ID NO: 86 - DNA, 20 bp, Artificial Sequence
  Synthesized oligonucleotide - primer for HPV type 69
ctacccgcagtaccaacctc

SEQ ID NO: 87 - DNA, 20 bp, Artificial Sequence
  Synthesized oligonucleotide - primer for HPV type 69
aactagcagtaggaggcaag

SEQ ID NO: 88 - DNA, 21 bp, Artificial Sequence
  Synthesized oligonucleotide - primer for HPV type 82
cattagcactgctgytactcc

SEQ ID NO: 89 - DNA, 21 bp, Artificial Sequence
  Synthesized oligonucleotide - primer for HPV type 82
ccykktgacaggakgttgctg

SEQ ID NO: 90 - DNA, 20 bp, Artificial Sequence
  Synthesized oligonucleotide - primer for HPV type 42
tgtgccactgcaacatctgg

SEQ ID NO: 91 - DNA, 23 bp, Artificial Sequence
  Synthesized oligonucleotide - primer for HPV type 42
ggatcctttttttctggcgttgt

SEQ ID NO: 92 - DNA, 22 bp, Artificial Sequence
  Synthesized oligonucleotide - primer for HPV type 74
acacgyagtactaacatgacwg

SEQ ID NO: 93 - DNA, 21 bp, Artificial Sequence
  Synthesized oligonucleotide - primer for HPV type 74
aaattkgcatagggattrggc